(19) United States
(12) Patent Application Publication
(10) Pub. No.: US 2015/0208975 A1
(43) Pub. Date: Jul. 30, 2015

(54) System and Method for Target Independent Neuromotor Analytics

(71) Applicant: Sync-Think, Inc., Boston, MA (US)

(72) Inventor: Jamshid Ghajar, Palo Alto, CA (US)

(21) Appl. No.: 14/552,190

(22) Filed: Nov. 24, 2014

Related U.S. Application Data
(60) Provisional application No. 61/933,202, filed on Jan. 29, 2014.

(51) Int. Cl. A61B 5/16; A61B 5/00; A61B 5/11

(52) U.S. Cl. CPC A61B 5/16; A61B 5/11; A61B 5/4884; A61B 5/7264

(57) ABSTRACT

A method is provided for testing a subject's cognitive performance by testing movements under the subject's voluntary control. The method includes prompting the subject to perform a plurality of iterations of a physical task. The physical task includes a movement of a first body part of the subject's body. For each iteration of the plurality of iterations of the physical task, one or more measurements corresponding to the movement of the first body part are obtained. A nominal path of the first body part is determined based on the measurements obtained from a first subset of the plurality of iterations of the physical task. A variability metric is generated by analyzing a plurality of measurements with respect to the nominal path. The variability metric is compared with a predetermined baseline to categorize the cognitive performance of the subject.

### RELATED APPLICATIONS

This application claims priority to U.S. Provisional Patent Application No. 61/933,202, filed Jan. 29, 2014, which is hereby incorporated by reference in its entirety.

### TECHNICAL FIELD

The disclosed embodiments relate generally to systems and methods of evaluating cognitive performance of a subject. More specifically, the disclosed embodiments relate to methods and systems for evaluating cognitive performance of a subject by testing movements under the subject's voluntary control.

### BACKGROUND

There are many circumstances in which it is desirable to evaluate cognitive perform of a subject (e.g., a person), especially when there is concern that the subject may be suffering from cognitive impairment. Voluntary control of motor function is dependent on cognitive function, specifically attention, such that assessment of voluntary motor function can be used to assess cognition/attention function. Formal neurological assessment of a subject can include many qualitative assessments of this cognitive-motor control linkage, such as evaluations of eye movement, pointing, rapid alternating movements and gait. However, these evaluations are qualitative and unreliable in the actual tests administered and in their interpretation.

Circumstances of cognitive impairment that benefit from assessment include acute medical emergencies affecting cognition—such as diabetic emergencies, environmental emergencies (e.g., heat stroke), poisoning (e.g., including intentional drug use and/or accident exposure to toxins), stroke, and/or traumatic brain injury (TBI)—as well as chronic impairments (e.g., dementia). In addition, law enforcement situations such as potential driving while intoxicated (DWI) infractions may merit evaluation of cognitive performance. Also, sleep deprivation leading to cognitive impairment is a concern for airline pilots, train conductors, and others whose cognitive performance is critical to the safety of themselves and others. In other circumstances, it is desirable to evaluate cognitive performance of a subject even when no cognitive impairment is suspected. For example, in some situations, subjects who are “cognitively ready” optimize their cognition using brain training exercises and games.

Unfortunately, existing methods of evaluating cognitive performance tend to be unreliable, effort dependent, invasive, expensive, overly qualitative, and/or inconvenient, and have learning effects. For example, in a hospital setting, types of cognitive evaluation generally fall into one of three categories. The first, a neurological physical exam, is performed by a highly trained medical provider (such as neurologist), and relies upon what is noticed or observed by the medical provider. Because a neurological physical exam generally requires a highly trained medical provider, it can be expensive and inconvenient for the subject, who must travel to the hospital and, in some circumstances, wait a prolonged period of time to meet with a specialist (e.g., the medical provider). Such tests are also qualitative because they depend on interpretation by the medical provider. The second category, brain imaging, is also expensive, requires interpretation by an expert, sometimes invasive, and yet is mainly sensitive to structural brain disruptions. The final category, mental status questionnaires, involves asking the subject questions about spatial and time orientation, such as “What is your name?”, “Where are you right now?” and “What time is it?” These questionnaires can be administered by lower-level medical providers (e.g., field medical personnel) and do not require a hospital or doctor's office. But such questionnaires are also highly qualitative and limited, leaving field medical personnel to rely on their intuition that the subject is “out of it.” Likewise, field sobriety tests (the standard cognitive test performed by law enforcement in potential DWI infractions) are similarly qualitative and inaccurate.

### SUMMARY

Accordingly, there is a need for accurate, quantitative systems and methods for evaluating cognitive performance of a subject that are reliable, accurate, inexpensive and convenient for the subject. Therefore, in accordance with some embodiments, a method, system, and computer-readable storage medium are proposed for cognitive evaluation of a subject.

To that end, some implementations provide a method for testing a subject's cognitive performance by testing movements under the subject's voluntary control. The method includes prompting the subject to perform a plurality of iterations of a physical task. The physical task includes a movement of a first body part of the subject's body. For each iteration of the plurality of iterations of the physical task, one or more measurements corresponding to the movement of the first body part are obtained. A nominal path of the first body part is determined based on the measurements obtained from a first subset of the plurality of iterations of the physical task. The first subset of the plurality of iterations of the physical task comprises some or all of said plurality of iterations of the physical task. The method further includes generating a variability metric by analyzing a plurality of measurements with respect to the nominal path. The plurality of measurements includes at least one measurement from each iteration of a second subset of the plurality of iterations of the physical task. The variability metric is compared with a predetermined baseline to categorize the cognitive performance of the subject.

In some embodiments, for each iteration of the plurality of iterations of the physical task, the one or more measurements corresponding to the movement of the first body part include two or more measurements corresponding to the movement of the first body part.

In some embodiments, the variability metric corresponds to variability of error with respect to the nominal path.

In some embodiments, the plurality of measurements includes a plurality of positional measurements, each positional measurement of the plurality of positional measurements being one of a position measurement, a velocity measurement, or an acceleration measurement, and each positional measurement including one or more positional values corresponding to the first body part.

In some embodiments, each positional value corresponds to a direction in accordance with a reference frame of measurement, the reference frame of measurement being one of a laboratory reference frame, a moving reference frame co-located with a target, or a moving reference frame co-located with a second body part.

In some embodiments, prompting the subject to perform the plurality of iterations of the physical task includes prompting the subject to move the first body part in a periodic manner with respect to the reference frame of measurement.

In some embodiments, prompting the subject to perform the plurality of iterations of the physical task includes prompting the subject to track a periodically moving target on a display. Said tracking is performed by the subject with a respective part of the subject's body and each iteration corresponds to a respective period of the periodic movement of the target.

In some embodiments, prompting the subject to perform the plurality of iterations of the physical task includes prompting the subject to walk a plurality of steps. Each step corresponds to a respective iteration of the plurality of iterations.

In some embodiments, prompting the subject to perform the plurality of iterations of the physical task includes prompting the subject to repeatedly reach with a respective hand toward an object. Each iteration corresponds to an instance of the subject reaching toward the object with the respective hand.

In some embodiments, the predetermined baseline corresponds to a predefined degree of variability in performing the physical task. In such embodiments, comparing the variability metric with the predetermined baseline to categorize the cognitive performance of the subject includes: categorizing the variability metric as indicative of a higher cognitive performance when the variability metric corresponds to a lower degree of variability in performing the physical task than the predefined degree of variability, and categorizing the variability metric as indicative of a lower cognitive performance when the variability metric corresponds to a higher degree of variability in performing the physical task than the predefined degree of variability.

In some embodiments, the method further includes prompting the subject to concurrently perform a second task while performing the physical task. The second task is a cognitive stressor.

In some embodiments, determining the nominal path of the first body part based on the measurements obtained from the first subset of the plurality of iterations of the physical task includes calculating a dynamical periodic orbit of motion. The dynamical periodic orbit of motion represents an average movement of the first body part over the first subset of the plurality of iterations of the physical task. In some embodiments, generating the variability metric includes calculating a value corresponding to an average distance between the measurements obtained from the second subset of the plurality of iterations of the physical task and the dynamical periodic orbit of motion.

In some embodiments, generating the variability metric by analyzing the plurality of measurements further includes producing a plurality of comparison values by, for each iteration of the second subset of the plurality of iterations of the physical task, comparing a respective measurement to a corresponding previous measurement from a previous iteration of the plurality of iterations to produce a respective comparison value. In some embodiments, the variability metric is then generated by aggregating the respective comparison values from each of the second subset of the plurality of iterations of the physical task.

In some embodiments, generating the variability metric by analyzing the plurality of measurements includes performing one or more of a return map analysis, a variance analysis, a covariance analysis, a principal component analysis, an independent component analysis, a K-means analysis, a medoid-based analysis, a dependency analysis, an entropy analysis, and a multi-resolution analysis.

In some embodiments, the predetermined baseline is based on at least one of: a variability range associated with a preselected group of control subjects, a demographic of the subject, and a variability metric for the subject generated from a previous test.

In another aspect of the present invention, to address the aforementioned limitations of cognitive evaluation techniques, some implementations provide a non-transitory computer readable storage medium storing one or more programs. The one or more programs comprise instructions, which when executed by an electronic device with one or more processors and memory, cause the electronic device to perform any of the methods provided herein.

In yet another aspect of the present invention, to address the aforementioned limitations of cognitive evaluation techniques, some implementations provide an electronic device. The electronic device includes one or more processors, memory, and one or more programs. The one or more programs are stored in memory and configured to be executed by the one or more processors. The one or more programs include an operating system and instructions that when executed by the one or more processors cause the electronic device to perform any of the methods provided herein.

### BRIEF DESCRIPTION OF THE DRAWINGS

FIG. 1 is a schematic diagram of a movement data acquisition environment, in accordance with some embodiments.

FIG. 2 is a conceptual block diagram illustrating a cognition diagnosis and training system, in accordance with some embodiments.

FIG. 3 is a detailed block diagram illustrating a cognition diagnosis and training system, in accordance with some embodiments.

FIGS. 4A-4D illustrate a flow diagram representing a method for evaluating cognitive performance of a subject by testing movements under the subject's voluntary control, in accordance with some embodiments.

FIGS. 5A-5C illustrate an example of a motion data acquisition scenario, in accordance with some embodiments

FIGS. 6A-6C illustrate another example of a motion data acquisition scenario, in accordance with some embodiments.

FIGS. 7A-7C illustrate yet another example of a motion data acquisition scenario, in accordance with some embodiments.

FIGS. 8A-8C illustrate motion analysis used to generated a variability metric, in accordance with some embodiments.

Like reference numerals refer to corresponding parts throughout the several views of the drawings.

### DETAILED DESCRIPTION OF EMBODIMENTS

Using methodologies described herein, it has been observed that with increasing cognitive and attention control, a subject's movements under voluntary control become less erratic or less variable. As a result, variability of the subject's motor function under voluntary control can be used to assess cognition and attention. To that end, in some embodiments, the methods, systems, and computer-readable storage media proposed herein evaluate cognitive status of a subject using variability of movements under the subject's voluntary control.

Pairing an action with anticipation of a sensory event is a form of attention that is crucial for an organism's interaction with the external world. The accurate pairing of sensation and action is dependent on timing and is called sensory-motor timing, one aspect of which is anticipatory timing. Anticipatory timing is essential to successful everyday living, not only for actions but also for thinking. Thinking or cognition can be viewed as an abstract motor function and therefore also needs accurate sensory-cognitive timing. Sensory-motor timing is the timing related to the sensory and motor coordination of an organism when interacting with the external world. Anticipatory timing is usually a component of sensory-motor timing and is literally the ability to predict sensory information before the initiating stimulus.

Anticipatory timing is essential for reducing reaction times and improving both movement and thought performance. Anticipatory timing only applies to predictable sensory-motor or sensory-thought timed coupling. The sensory modality (e.g., visual, auditory etc.), the location, and the time interval between stimuli, must all be predictable (e.g., constant, or consistent with a predictable pattern) to enable anticipatory movement or thought.

Without reasonably accurate anticipatory timing, a person cannot catch a ball, know when to step out of the way of a moving object (e.g., negotiate a swinging door), get on an escalator, comprehend speech, concentrate on mental tasks or handle any of a large number of everyday tasks and challenges. Even walking requires responding to a large number of stimuli (e.g., the level of the ground, the shifting of weight) with accurate anticipatory timing. The inability to do so leads to conditions such as ataxia (e.g., variable gait movement). This capacity for anticipatory timing can become impaired with sleep deprivation, aging, alcohol, drugs, hypoxia, infection, clinical neurological conditions including but not limited to attention deficit hyperactivity disorder (ADHD), schizophrenia, autism and brain trauma (e.g., a concussion). For example, brain trauma may significantly impact a person's cognition timing, one aspect of which is anticipatory timing. Sometimes, a person may appear to physically recover quickly from brain trauma, but have significant problems with concentration and/or memory, as well as having headaches, being irritable, and/or having other symptoms as a result of impaired anticipatory timing. In addition, impaired anticipatory timing may cause the person to suffer further injuries by not having the timing capabilities to avoid accidents.

Anticipatory timing in cognition and movement are controlled by the same core brain circuits. Variability in anticipatory timing produces imprecise movements and disrupted thinking, such as difficulty in concentration, memory recall, and carrying out both basic and complex cognitive tasks. Such variability leads to longer periods of time to successfully complete tasks and also leads to more inaccuracy in the performance of such tasks. Therefore diagnosis and training (e.g., therapy) can be performed for anticipatory timing difficulties in the motor and cognitive domains using motor reaction times and accuracy. In particular, both the reaction time and accuracy of a subject's movements can be measured. As discussed below, these measurements can be used for both diagnosis and training (e.g., therapy).

Accordingly, in some embodiments, such variability is measured to determine whether a person suffers impaired anticipatory timing. More specifically, in some implementations, a subject is prompted to repeatedly perform a physical task that includes reacting to stimuli, be they prompted stimuli (e.g., having the subject track a moving ball on a display with her finger, in which case the movement of the ball is the stimuli) or natural stimuli (e.g., having the subject walk in a straight line, in which case the stimuli include mechanical stimuli, such as shifting weight and the level of the ground). Because the physical task is performed repeatedly, in some circumstances, a healthy subject develops a sense of the mechanics of the task, which serves to reduce variability of the subject's movements in performing the task. Moreover, the movement is repetitive in some manner, and in some embodiments variability of the subject's movement is analyzed without regard to position information of the stimuli (e.g., without regard to the position of the ball being tracked). For example, in some embodiments, a nominal path of movement is determined using measurements from some or all of the repetitions of the physical task, and a variability metric is calculated with respect to the nominal path.

A lower degree of variability in performing repetitive movements under a subject's voluntary control is generally associated with higher cognitive performance of the subject. This stands in contrast to autonomic functions of the subject (e.g., functions not under the subject's voluntary control, such as the beating of the subject's heart), in which increased variability can indicate a higher degree of health.

Reference will now be made in detail to various implementations, examples of which are illustrated in the accompanying drawings. In the following detailed description, numerous specific details are set forth in order to provide a thorough understanding of the present disclosure and the described implementations herein. However, implementations described herein may be practiced without these specific details. In other instances, well-known methods, procedures, components, and mechanical apparatus have not been described in detail so as not to unnecessarily obscure aspects of the implementations.

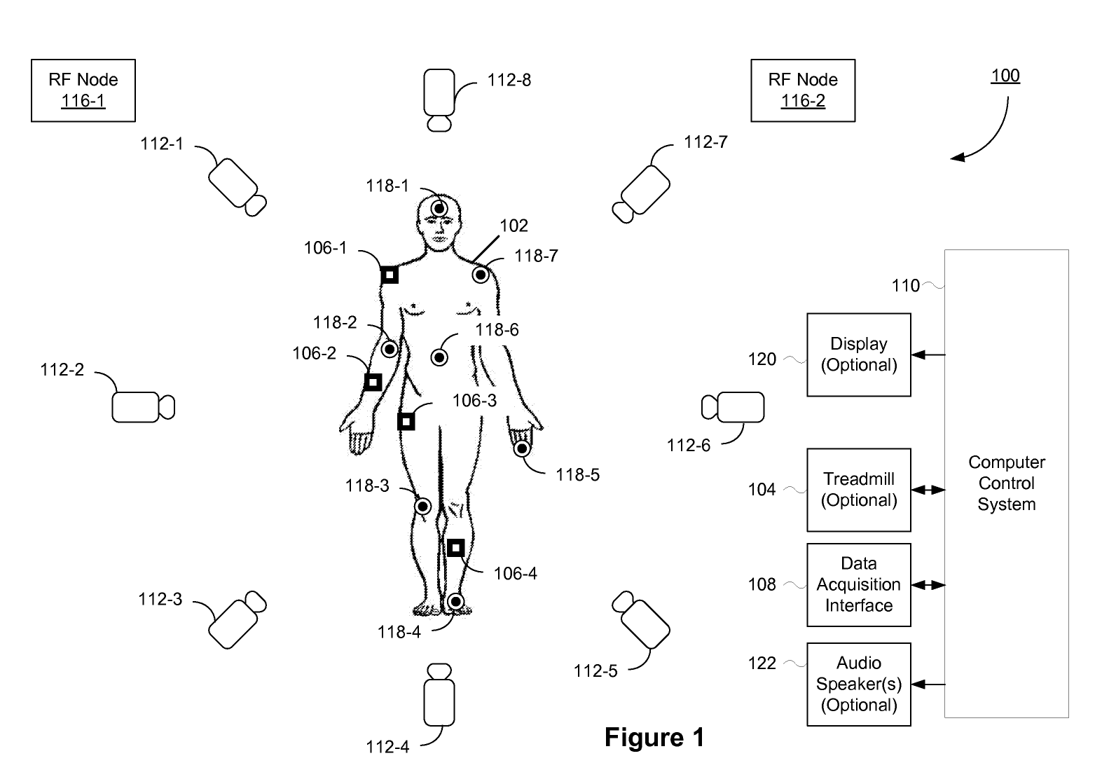

FIG. 1 is a schematic diagram of a motion data acquisition environment 100, in accordance with some embodiments. In some embodiments, during a motion data acquisition scenario performed within motion data acquisition environment 100, a computer control system 110 prompts a subject 102 to repeatedly perform a physical task that includes movement of a particular part of subject 102's body. For example, in some embodiments, computer control system 110 prompts subject 102 to walk on a treadmill 104 (e.g., the physical task is to take a step, so that walking comprises repetition of the physical task). For each iteration of the physical task, computer control system 110 obtains one or more measurements corresponding to the movement of the particular part of subject 102's body. For example, when the physical task is to walk on treadmill 104, in some circumstances, computer control system 110 obtains one or more measurements of a respective foot-position of subject 102 for each step. Alternatively, computer control system 110 obtains one or more measurements of the position of both feet during each step, or one or more measurements of the position of a respective knee, or both knees, etc. As described below, computer control system 110 uses the one or more measurements to evaluate cognitive performance of subject 102 by determining a nominal path of the particular part of subject 102's body using the one or more measurements and analyzing variability of the one or more measurements with respect to the nominal path.

Computer control system 110 is configured to obtain the measurements of the particular part of subject 102's body. For example, as shown in FIG. 1, in some embodiments, computer control system 110 interfaces with one or more motion sensors 106 (e.g., motion sensor 106-1 through motion sensor 106-4) through a data acquisition interface 108. In this manner, computer control system 110 receives positional information about subject 102, including positional information about the particular part of subject 102's body from which positional measurements are directly obtained or can be inferred. In various embodiments, motion sensors 106 include inertial elements (e.g., accelerometers)—such as micro-electro-mechanical system (MEMS) gyroscopes—and/or radio frequency (RF) positioning elements, or a combination thereof. When motion sensors 106 include RF positioning elements, the positional measurements variously include relative positional measurements between two or more motion sensors 106 and/or absolute positional measurements (e.g., positional measurements with respect to a laboratory reference frame) determined by locating a respective motion sensor 106 with respect to one or more RF nodes 116 (e.g., RF node 116-1 and 116-2), whose locations are known within the laboratory. Such techniques, e.g., establishing one or more RF element locations with respect to one or more RF nodes, are sometimes called “Indoor Positioning System” (IPS) techniques.

In some embodiments, motion data acquisition environment 100 is part of a laboratory setting, such as a scientific laboratory or a medical examination room. Alternatively, motion data acquisition environment 100 is not part of a laboratory setting. For example, in some embodiments, the one or more motion sensors 106 are included in a portable device, such as a portable multifunction device (e.g., a smart-phone) or a remote control, thus freeing the systems and methods described herein to be used in any environment, such as a home or in a public place. In some embodiments, the one or more motion sensors 106 are accessories to another device, such as a smart-phone or an exercise apparatus. As an example, a set of ankle bands, each with one or more motion sensors 106 can be used with an off-the-shelf treadmill to effectively comprise a motion data acquisition environment 100. This is particularly true when the treadmill is enabled with various smart features such as the ability to execute a computer program (e.g., an application or an “app”) that accompanies the ankle bands (e.g., the treadmill includes one or more processors that, together with the computer program, can serve the role of computer control system 110). In this manner, subject 102 may effectively transform her own home gymnasium into a motion data acquisition environment 100.

In some embodiments, when motion data acquisition environment 100 is part of a laboratory setting, computer control system 110 interfaces with one or more cameras 112 through data acquisition interface 108. In some implementations, the one or more cameras 112 receive signals from one or more optical markers 118 that are placed at respective locations on subject 102's body. Variously, optical markers 118 include passive markers (e.g., disks of a reflective material that reflect ambient light) and/or active markers (e.g., markers that include a light source such as a light emitting diode (LED)). In some embodiments, the use of optical markers is obviated by using feature-recognition analysis. For example, in some embodiments, motion data acquisition environment 100 includes a non-transitory computer readable storage medium (e.g., memory) storing instructions that, when executed by one or more processors on computer control system 110, cause computer control system 110 to recognize a particular part of subject 102's body (e.g., one of her feet) and determine positional information corresponding to the particular part of subject 102's body. The positional information is then used to calculate positional measurements for the particular part of the subject 102's body. When the one or more cameras 112 comprise a plurality of cameras 112 (e.g., a number of cameras between 2 and 48, or more), the positional information from the plurality of cameras 112 can be used to calculate (e.g., triangulate) three-dimensional positional measurements of the particular part of subject 102's body.

In some embodiments, the one or more cameras 112 are digital video cameras that record images at a rate of at least 200 Hertz (Hz), or equivalently, record at least 200 images per second. In some embodiments, the one or more cameras 112 are digital video cameras that record images at a rate of 500 Hertz (Hz), or equivalently, record 500 images per second.

As mentioned above, computer control system 110 is coupled with (e.g., configured to interface with) the one or more motion sensors 106 and the one or more cameras 112 through data acquisition interface 108, which can include wireless or wired components. Computer control system 110 is optionally coupled with a display 120, which can be a computer monitor, projector screen, or other display device (e.g., a display device for an exercise apparatus). Display 120 presents visual stimuli. Computer control system 110 is also optionally coupled with one or more audio speaker(s) 122 for presenting audio stimuli. In some embodiments, computer control system 110 prompts subject 102 using display 120 and/or audio speaker(s) 122 to repeatedly perform the physical task (e.g., display 120 and/or audio speaker(s) 122 can be used to deliver, to subject 102, the prompt to repeatedly perform the physical task). In some embodiments, subject 102 uses display 120 and/or the one or more audio speaker(s) 122 to perform a second task, which, in some circumstances, is a cognitive stressor.

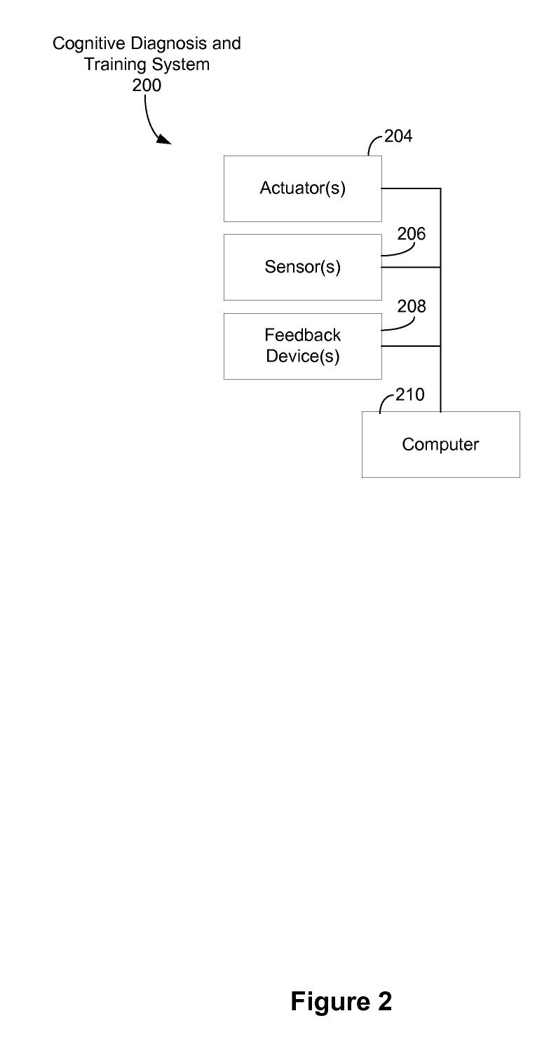

FIG. 2 illustrates a conceptual block diagram of a cognition diagnosis and training system 200, in accordance with some embodiments. Cognitive diagnosis and training system 200 includes computer 210 (e.g., computer control system 110) coupled to one or more actuators 204, and one or more sensors 206. In some embodiments, system 200 includes one or more feedback devices 208 (e.g., when system 200 is configured for use as a cognitive training system).

In some embodiments, feedback is provided to the subject via the actuators 204. In some embodiments, actuators 204 include a display device for presenting visual stimuli to a subject, audio speakers for presenting audio stimuli, mechanical actuators (e.g., a treadmill) for presenting mechanical stimuli to the subject, or a combination of the aforementioned, or one or more other devices for producing or presenting sequences of stimuli to a subject. Sensors 206 include one or more motion sensors 106, one or more cameras 112 shown in FIG. 1, or both. System 200 also includes, in some circumstances, additional sensors 206 that are, optionally, mechanical, electrical, electromechanical, auditory (e.g., microphone), and/or other type of sensors (e.g., a frontal brain electroencephalograph, known as an EEG). The primary purpose of sensors 206 is to detect responses by a subject (e.g., subject 102 in FIG. 1) to sequences of stimuli presented by actuators 204. Such responses can include physical movements of the subject that are performed in response to a prompt to perform a physical task. For example, when the subject is prompted to walk on a treadmill (e.g., either because the treadmill displayed a visual stimulus to walk, or because the treadmill simply starts moving, which is an example of a mechanical stimulus), a foot position of the subject can be considered a “response.”

Some types of sensors produce large amounts of raw data, only a small portion of which is indicative of the subject's response. In such systems, computer 210 contains appropriate filters and/or software procedures for analyzing the raw data so as to extract “sensor signals” indicative of the subject's response to the stimuli. For example, as shown in FIG. 1, in some embodiments, cameras 112 collect full images of the subject, and appropriate filters and/or software procedures analyze the images to produce positional measurement of a particular part of the subject's body (e.g., a part to which an optical marker 118 is affixed).

In embodiments in which sensors 206 includes an electroencephalograph (EEG), the relevant sensor signal from the EEG may be a particular component of the signals produced by the EEG, such as the contingent negative variation (CNV) signal or the readiness potential signal.

Feedback devices 208 are, optionally, any device appropriate for providing feedback to a subject (e.g., subject 102 in FIG. 1). In some embodiments, feedback devices 208 provide real-time performance information to the subject corresponding to measurement results, which enables the subject to try to improve his/her anticipatory timing performance. In some embodiments, the performance information provides positive feedback to the subject when the subject's responses (e.g., to sequences of stimuli) are within a normal range of values. In some embodiments, the performance information provides positive feedback to the subject when the subject's responses reflect improved anticipatory timing performance relative to earlier performance by the subject, even if the subject's current anticipatory timing performance is not within a normal range. In some embodiments, the one or more feedback devices 208 may activate the one or more actuators 204 in response to positive performance from the subject, such as by changing the color of the visual stimuli or changing the pitch or other characteristics of the audio stimuli, or increasing the speed of the mechanical stimuli.

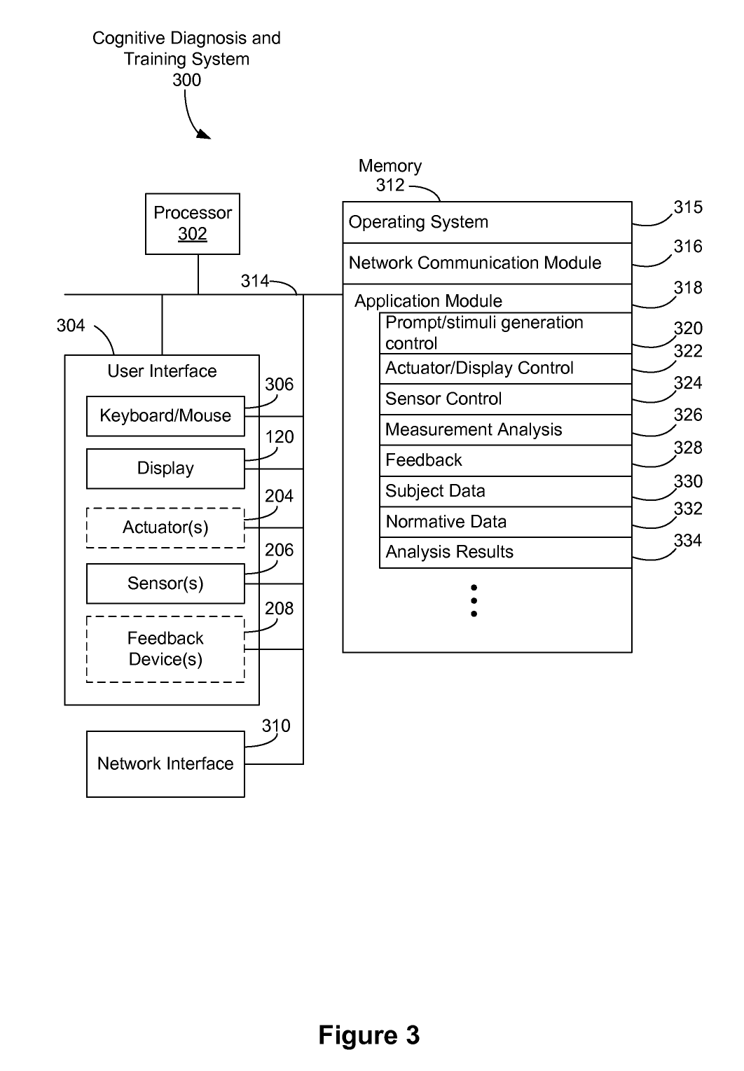

FIG. 3 is a block diagram of a cognition diagnosis and training (or remediation) system 300 in accordance with some embodiments. The system includes one or more processors 302 (e.g., CPUs), user interface 304, memory 312, and one or more communication buses 314 for interconnecting these components. In some embodiments, the system includes one or more network or other communications interfaces 310, such as a network interface for conveying testing or training results to another system or device. The user interface 304 includes one or more actuators 204 and one or more sensors 206, and, in some embodiments, also includes one or more feedback devices 208. In some embodiments, the user interface 304 further includes additional computer interface devices such as keyboard/mouse 306 and display 120. In some embodiments, display 120 is coupled with one or more actuators 204.

In some implementations, memory 312 includes a non-transitory computer readable medium, such as high-speed random access memory and/or non-volatile memory (e.g., one or more magnetic disk storage devices, one or more flash memory devices, one or more optical storage devices, and/or other non-volatile solid-state memory devices). In some implementations, memory 312 includes mass storage that is remotely located from processing unit(s) 302. In some embodiments, memory 312 stores an operating system 315 (e.g., Microsoft Windows, Linux or Unix), an application module 318, and network communication module 316.

In some embodiments, application module 318 includes prompt/stimuli generation control module 320, actuator/display control module 322, sensor control module 324, measurement analysis module 326, and, optionally, feedback module 328. Prompt/stimuli generation control module 320 generates prompts and or sequences of stimuli (e.g., used to prompt a subject to perform a physical task), as described elsewhere in this document. Actuator/display control module 322 produces or presents the sequences of stimuli to a subject. Sensor control module 324 receives sensor signals and, where appropriate, analyzes raw data in the sensor signals so as to extract sensor signals indicative of the subject's (e.g., subject 102 in FIG. 1) response to the stimuli. In some embodiments, sensor control module 324 includes instructions for controlling operation of sensors 206. Measurement analysis module 326 analyzes the sensor signals (e.g., motion sensors 106 and/or cameras 112) to produce measurements and analyses, as discussed elsewhere in this document. For example, measurement analysis module 326 analyzes measurements from a plurality of iterations of a physical task performed by a subject to (i) determine a nominal path of a first body part of the subject, and (ii) generate a variability metric by analyzing the measurements with respect to the nominal path. Feedback module 328, if included, generates feedback signals for presentation to the subject through the one or more actuators or feedback devices.

In some embodiments, application module 318 furthermore stores subject data 330, which includes measurement data for a subject, analysis results 334, and the like. For example, in some implementations analysis results 334 includes a baseline variability metric for the subject (e.g., a baseline obtained during a previous test performed when the subject was considered “healthy”). In some embodiments, application module 318 stores normative data 332. In some implementations, normative data 332 includes measurement data from one or more control groups of subjects, and/or analysis results based on the measurement data from the one or more control groups.

Still referring to FIG. 3, in some embodiments, sensors 206 include one or more or more digital video cameras (e.g., cameras 112, FIG. 1) configured to record images that include one or more optical markers (e.g., optical markers 118, FIG. 1), operating at a picture update rate of at least 200 Hertz (Hz). In some embodiments, the one or more digital video cameras are infrared cameras, while in other embodiments, the cameras operate in other portions of the electromagnetic spectrum. In some embodiments, the resulting video signal is analyzed by processor 302, under the control of measurement analysis module 326, to determine the position(s) of the optical markers 118, and the timing of when the position(s) were recorded. In some embodiments, sensors 206 include motion sensors (e.g., motion sensors 106, FIG. 1).

In some embodiments, not shown, the system shown in FIG. 3 is divided into two systems, one which tests a subject and collects data, and another which receives the collected data, analyzes the data (e.g., using measurement analysis module 326) and generates one or more corresponding reports.

### Diagnostic Methods

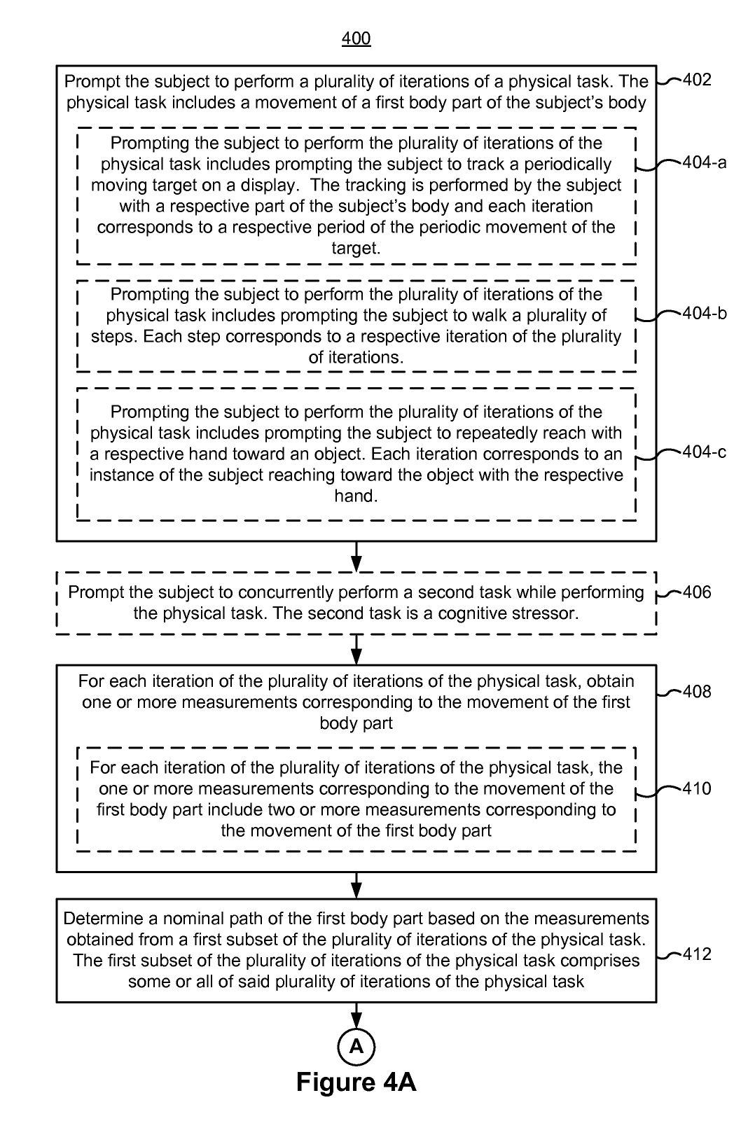

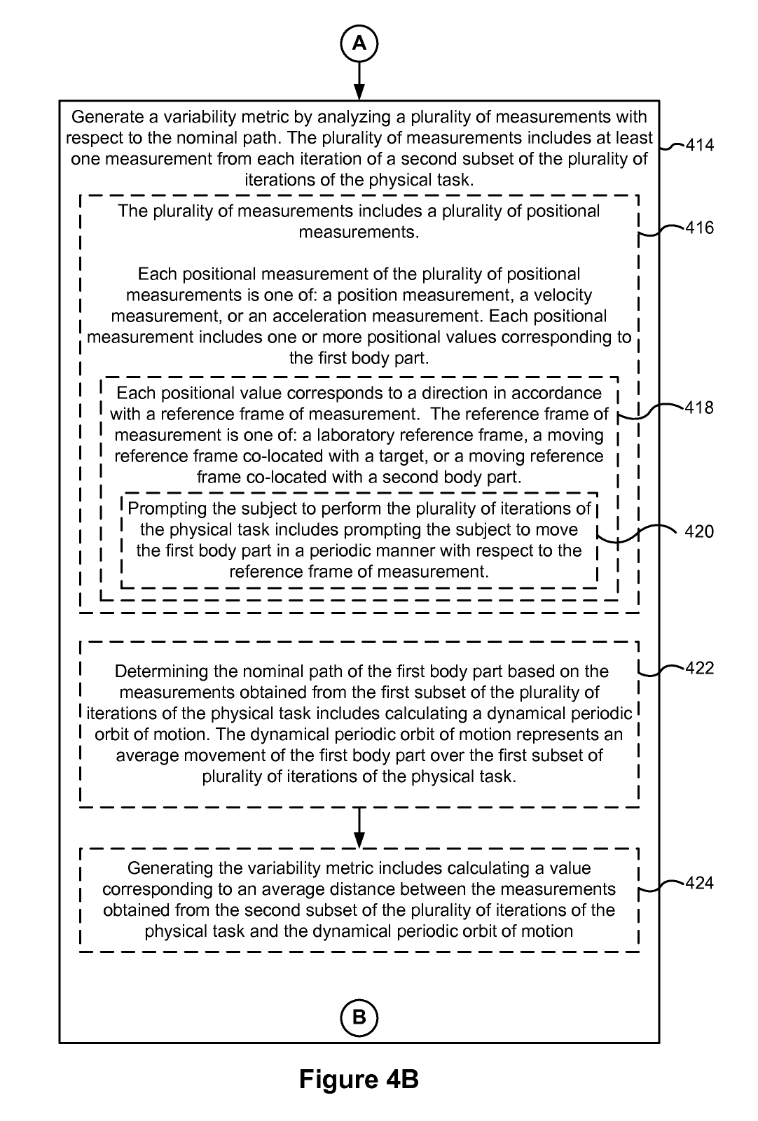

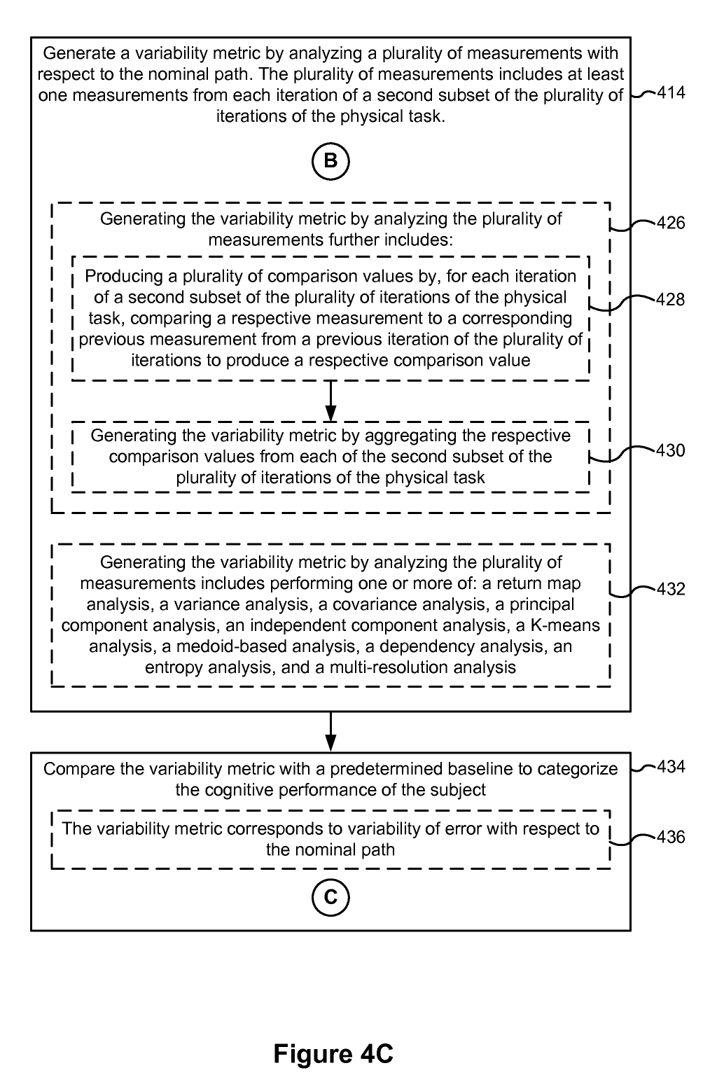

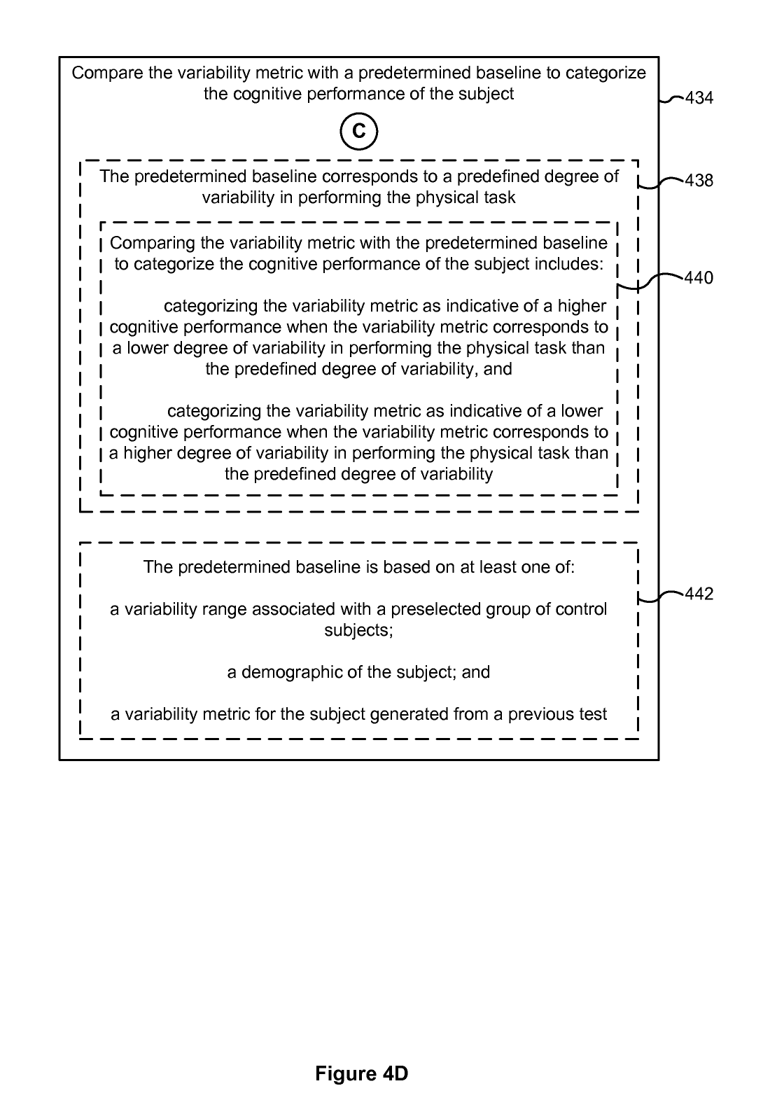

FIGS. 4A-4D illustrate a flow diagram representing a method 400 for evaluating cognitive performance of a subject by testing movements under the subject's voluntary control, in accordance with some embodiments. The method is, optionally, governed by instructions that are stored in a computer memory or non-transitory computer readable storage medium (e.g., memory 312 in FIG. 3) and that are executed by one or more processors (e.g., processor 302) of one or more systems, such as, but not limited to, system 300, computer control system 110, or system 200. The computer readable storage medium may include a magnetic or optical disk storage device, solid state storage devices such as Flash memory, or other non-volatile memory device or devices. The computer readable instructions stored on the computer readable storage medium may include one or more of: source code, assembly language code, object code, or other instruction format that is interpreted by one or more processors. In various implementations, some operations in each method may be combined and/or the order of some operations may be changed from the order shown in the figures. Also, in some implementations, operations shown in separate figures and/or discussed in association with separate methods may be combined to form other methods, and operations shown in the same figure and/or discussed in association with the same method may be separated into different methods. Moreover, in some implementations, one or more operations in the method are performed by modules of cognitive diagnosis and training system 200 (FIG. 2) and/or the system shown in FIG. 3, including, for example, operating system processor 302, user interface 304, memory 312, network interface 310, and/or any sub-modules thereof. For ease of explanation, at least some aspects of method 400 are described with reference to cognitive diagnosis and training system 200 (hereinafter “system 200”), or system 100, or system 300, or a combination thereof.

A cognitive diagnosis system (e.g., cognitive diagnosis and training system 200) prompts (402) the subject to perform a plurality of iterations of a physical task (e.g., using prompt/stimuli generation control module 320, FIG. 3). The physical task includes a movement of a first body part of the subject's body.

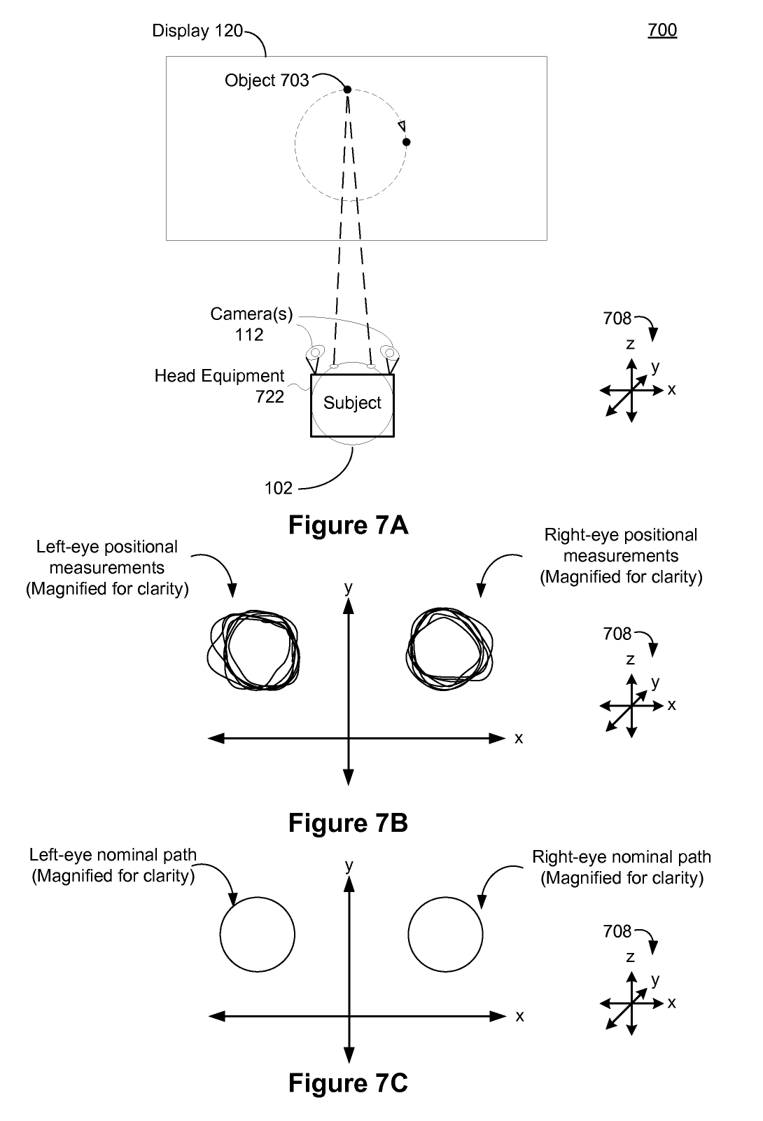

In some embodiments, prompting the subject to perform the plurality of iterations of the physical task includes (404-a) prompting the subject to track a periodically moving target on a display. The tracking is performed by the subject with a respective part of the subject's body (e.g., a finger, a foot, or the subject's eyes) and each iteration corresponds to a respective period of the periodic movement of the target (e.g., one period is a complete traversal of the target around a predefined path on the display). In some embodiments, the respective part of the subject's body is the first body part. As an example, a motion data acquisition scenario is described with reference to FIG. 7A-7C in which a subject is prompted to track with her eyes an object moving periodically on a display.

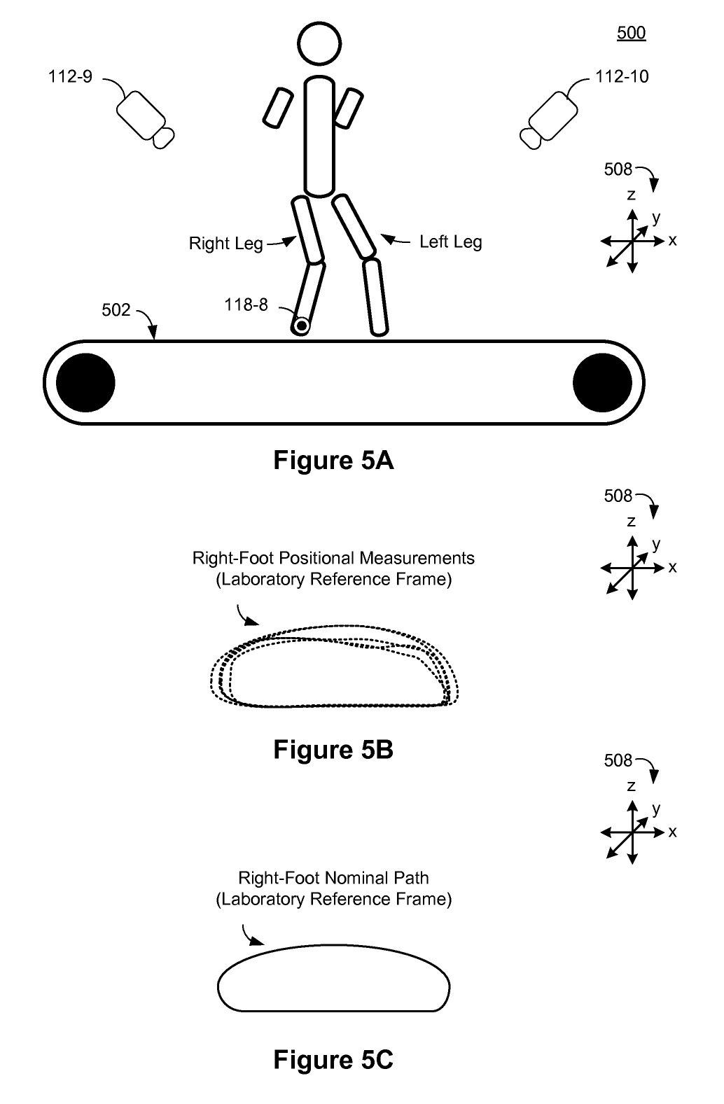

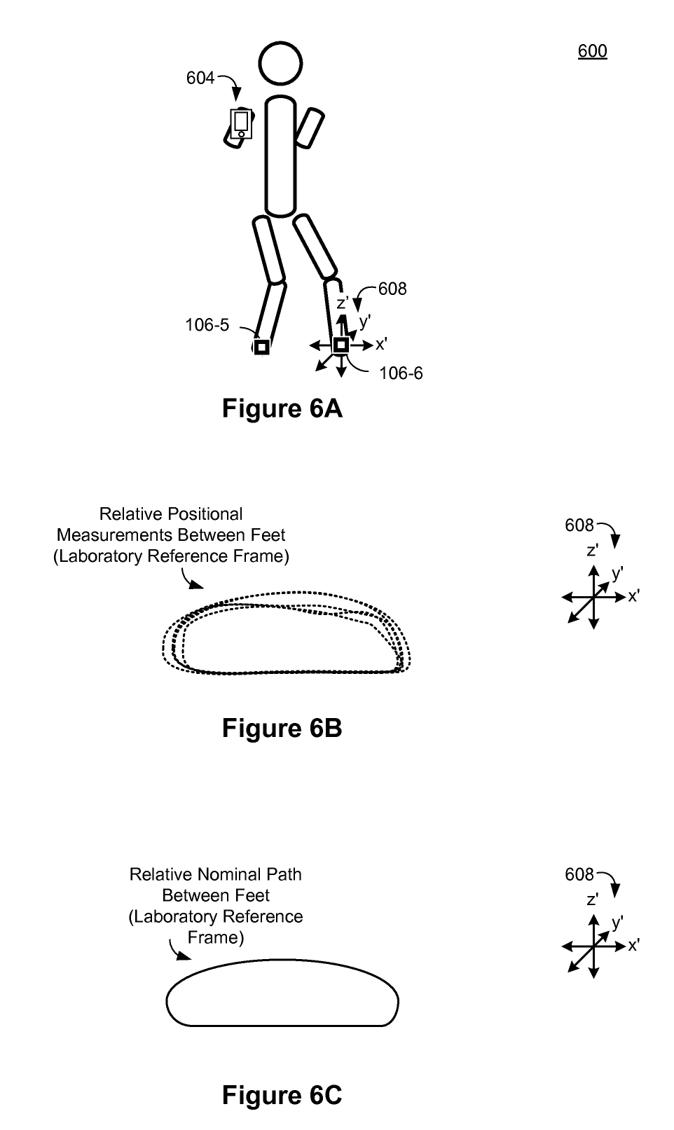

In some embodiments, prompting the subject to perform the plurality of iterations of the physical task includes (404-b) prompting the subject to walk a plurality of steps. Each step, or each pair of steps (one with each foot), corresponds to a respective iteration of the plurality of iterations. A motion data acquisition scenario, in which a subject is prompted to walk a plurality or sequence of steps on a treadmill, is described with references to FIGS. 5A-5C. Another motion data acquisition scenario, in which a subject is prompted to walk a plurality or sequence of steps without the use of a treadmill, is described with references to FIGS. 6A-6C.

In some embodiments, prompting the subject to perform the plurality of iterations of the physical task includes (404-c) prompting the subject to repeatedly reach with a respective hand toward an object. Each iteration corresponds to an instance of the subject reaching toward the object with the respective hand.

In some embodiments, the cognitive diagnosis system prompts (406) the subject to concurrently perform a second task while performing the physical task. The second task is a cognitive stressor. For example, in some embodiments, the second task is a game or a puzzle that the subject interacts with on a display (e.g., the subject is asked to play Soduku, a cognitive stressor, while walking on a treadmill, which is a physical task). In some embodiments, the second task is a cognitive training game, such as an n-back game, that is designed to train the subject to increase her concentration and/or working memory. In some embodiments, the second task is a second physical task. For example, when the physical task is to walk at a constant rate on a treadmill, the second task can include reaching for an object. It should be understood, however, that the second task need not be a purely mental task or a purely physical task, but may be a combination of the two.

For each iteration of the plurality of iterations of the physical task, the cognitive diagnosis system obtains (408) one or more measurements corresponding to the movement of the first body part (e.g., using sensors 106, 206 or 306, such as cameras 112, optical markers 118, and/or motion sensors 106, FIG. 1, together with sensor control module 324, FIG. 3). In some embodiments, for each iteration of the plurality of iterations of the physical task, the one or more measurements corresponding to the movement of the first body part include (410) two or more measurements corresponding to the movement of the first body part (or three or more, or four or more, etc.). In some circumstances, the movement of the first body part during a respective iteration is referred to as a respective “orbit.”

In some embodiments, measurements are obtained “continuously,” meaning that the measurements are obtained at a predefined sampling rate that is substantially faster than a frequency at which iterations are performed (e.g., an inverse of a period, or duration, of an average iteration). For example, in some embodiments, the subject is prompted to track, with her finger, a periodically moving target on a display (see operation 404) moving at a frequency of 1 Hertz (Hz). In this example, in some embodiments, cognitive diagnosis system obtains “continuous” measurements of a position of the subject's finger at a sampling rate of 250 Hz. Such continuous measurements are an example in which the measurements are decoupled from the subject's movement. Conversely, when a subject is tasked with walking on a treadmill, in some implementations, the location of the subject's foot strike, foot lift, and foot zenith are measured; this is an example in which measurements are coupled to the subject's movements.

The cognition diagnosis system (e.g., system 200 or system 300) determines (412) a nominal path (e.g., a nominal orbit) of the first body part based on the measurements obtained from a first subset of the plurality of iterations of the physical task (e.g., using measurement analysis module 326, FIG. 3). The first subset of the plurality of iterations of the physical task comprises some or all of said plurality of iterations of the physical task. In some embodiments, the first subset of the plurality of iterations of the physical task comprises all of said plurality of iterations that occur after a predefined period. The predefined period can be specified as or identified by a predefined number of iterations or, alternatively, a predefined amount of time. The predefined period is sometimes referred to as a “training period,” and it is optionally provided in order to allow the subject to acclimate to performance of the physical task. In some embodiments, the first subset of the plurality of iterations of the physical task comprises a single iteration of the physical task. For example, as discussed below with reference to operation 428-432, in some embodiments, the nominal path is the path of the first body part taken in a previous iteration (i.e., the previous iteration is the iteration occurring immediately prior to a current iteration). In this example, the path of the first body part taken in the previous iteration is compared to the path of the first body part taken in the current iteration to generate a variability metric (see operation 414). In some other embodiments, the first subset of the plurality of iterations of the physical task comprises multiple iterations of the physical task.

The cognition diagnosis system (e.g., system 200 or system 300) generates (414) a variability metric by analyzing a plurality of measurements with respect to the nominal path (e.g., using measurement analysis module 326, FIG. 3). The plurality of measurements includes at least one measurement from each iteration of a second subset of the plurality of iterations of the physical task. In some embodiments, the second subset of the plurality of iterations of the physical task comprises all of said plurality of iterations that occur after the predefined period, or “training period,” as discussed above. In some embodiments, the first subset of the plurality of iterations of the physical task and the second subset of the plurality of iterations of the physical task are identical (e.g., both subsets comprise all of said plurality of iterations that occur after the predefined period).

In some embodiments, the plurality of measurements includes (416) a plurality of positional measurements. Each positional measurement of the plurality of positional measurements is one of: a position measurement, a velocity measurement (e.g., inferred using two or more position measurements of the first body part), or an acceleration measurement (e.g., inferred using three or more position measurements of the first body part, or measured directly using an accelerometer). In some embodiments, the one or more measurements correspond to generalized coordinates. For example, in some embodiment, the plurality of measurements also includes one or more angular measurements, such as a measurement of a respective angle between body parts (e.g., an angle between the first body part, a second body part, and a joint coupling the first body part and the second body part), a measurement of an angular velocity (e.g., computed or inferred using two or more measurements of a respective angle between body parts), and/or a measurement of an angular acceleration (e.g., computed or inferred using three or more measurements of a respective angle between body parts). Each positional measurement includes one or more positional values corresponding to the first body part. Each positional value corresponds (418) to a direction in accordance with a reference frame of measurement. For example, in some embodiments, each of the one or more positional measurements includes an ordered-tuple of (x, y, z, t) values, in which (x, y, z) are measurements along Cartesian measurement axes (sometimes called Cartesian axes) in a respective reference frame of measurement (sometimes called a frame of reference), and t is time. Alternatively, in some embodiments, positional measurements are determined with respect to only one direction (e.g., only an x-component of motion), or only two directions (e.g., only an x- and a y-component of motion, thereby forgoing measurement in the z-direction). Variously, the reference frame of measurement is one of: a laboratory reference frame, a moving reference frame co-located with a target (e.g., target 703, FIG. 7A), or a moving reference frame co-located with a second body part (e.g., in FIG. 5A, the subject's left foot is the first body part and the subject's right foot is the second body part).

In some embodiments, the first body part and the second body part are anatomically equivalent. For example, the first body part is a respective foot, finger, eye, elbow, or the like, and the second body part is the other foot, finger, eye, elbow. Alternatively, in some embodiments, the first body part is a foot, finger, eye, elbow, or the like, and the second body part is an anatomically distinct body part (e.g., the first body part is a foot and the second body part is a center of mass of the subject, or a finger, eye, or elbow of the subject).

In some embodiments, prompting the subject to perform the plurality of iterations of the physical task includes (420) prompting the subject to move the first body part in a periodic manner with respect to the reference frame of measurement. In some embodiments, the prompt is considered a prompt to move the first body part in a periodic manner when perfect execution of the physical task would result in periodic movement of the first body part (e.g., with respect to a respective reference frame). In such embodiments, or in some circumstances, greater variability in the subject's movements (indicating greater deviation from periodic movement) is indicative of a decreased cognitive status.

In some embodiments, determining the nominal path of the first body part based on the measurements obtained from the first subset of the plurality of iterations of the physical task includes (422) calculating a dynamical periodic orbit of motion. The term “dynamical periodic orbit of motion” is intended to indicate that the nominal path is, in some embodiments, a path of a state of the first body part through a state-space (e.g., a phase-space that includes dimensions corresponding to position, velocity, acceleration, and/or momentum, rather than solely one or more position dimensions). More generally, in some embodiments, the nominal path is a path of a state of the first body part through a state-space of generalized coordinates (e.g., generalized coordinates comprise a set of parameters that define a configuration of the first body part). In some embodiments, the dynamical periodic orbit of motion represents an average movement of the first body part (e.g., in position-space or phase-space) over the first subset of the plurality of iterations of the physical task. In some embodiments, generating the variability metric includes (424) calculating a value corresponding to an average distance between the measurements obtained from the second subset of the plurality of iterations of the physical task (e.g., measurements belonging to the state-space) and the dynamical periodic orbit of motion.

In some embodiments, generating the variability metric by analyzing the plurality of measurements further includes (426) producing (428) a plurality of comparison values by, for each iteration of a second subset of the plurality of iterations of the physical task, comparing a respective measurement to a corresponding previous measurement from a previous iteration of the plurality of iterations to produce a respective comparison value. Such an analysis is sometimes referred as a “return map” analysis. In some embodiments, the system 200 generates (430) the variability metric by aggregating the respective comparison values from each of the second subset of the plurality of iterations of the physical task.

In some embodiments, generating the variability metric by analyzing the plurality of measurements includes (432) performing one or more of (or a combination of): a return map analysis, a variance analysis, a covariance analysis, a principal component analysis, an independent component analysis, a K-means analysis, a medoid-based analysis, a dependency analysis (e.g., a “Spearman's rho” analysis or a Kendall tau analysis), an entropy analysis, and a multi-resolution (e.g., wavelet) analysis.

The cognition diagnosis system (e.g., system 200 or system 300) compares (434) the variability metric with a predetermined baseline to categorize the cognitive performance of the subject. In some embodiments, the variability metric corresponds (436) to variability of error with respect to the nominal path. In some embodiments, system 200 compares a stressed variability metric (e.g., a metric obtained while the subject is concurrently performing a cognitive stressor, see operation 406) with an un-stressed variability metric (for the same subject) to determine whether the subject has a stress-sensitive impairment (e.g., post-traumatic stress disorder (PTSD), early-stage dementia, etc.). For example, in accordance with some implementations, if the stressed variability metric is significantly higher than the un-stressed variability metric (e.g., if the stressed variability metric exceeds the variability metric by more than a predefined threshold), that result indicates that the subject suffers from a stress-sensitive impairment (e.g., PTSD, early-stage dementia).

In some embodiments, the predetermined baseline corresponds to a predefined degree of variability in performing the physical task. In some embodiments, comparing the variability metric with the predetermined baseline to categorize the cognitive performance of the subject includes (440):
categorizing the variability metric as indicative of a higher cognitive performance when the variability metric corresponds to a lower degree of variability in performing the physical task than the predefined degree of variability, and
categorizing the variability metric as indicative of a lower cognitive performance when the variability metric corresponds to a higher degree of variability in performing the physical task than the predefined degree of variability.

In some circumstances, a lower degree of variability in performing tasks under voluntary control indicates a higher cognitive status of the subject. This stands in contrast to certain autonomic bodily functions, such as heart rate, in which a higher degree of variability can indicate, in some circumstances, a higher degree of performance (e.g., a more elastic response to stimuli and/or a greater dynamic range). For example, a higher degree of heart-rate variability can, in some circumstances, indicate that the subject's heart is better able to respond to stressors, whereas a higher degree of variability in tracking a target can, in some circumstances, be indicative of a depressed cognitive status (e.g., a lower cognitive performance) as compared to a subject with a lower degree of variability in performing the same task.

In some embodiments, the predetermined baseline is based on at least one of: a variability range associated with a preselected group of control subjects (e.g., stored in normative data 332, FIG. 3), a demographic of the subject (e.g., stored in normative data 332, FIG. 3), and a variability metric for the subject generated from a previous test (e.g., stored in analysis result 334).

Thus, method 400 provides a fast, convenient, and cost effective manner through which to evaluate a subject's cognitive status. Since method 400 can easily be implemented on a portable apparatus such as a laptop or tablet computer (e.g., with measurement accessories), field medical personal (e.g., emergency medical technicians) can utilize various implementations of method 400 in order to evaluate a patient's cognitive status during a medical emergency. For some of the same reasons, method 400 can also be utilized by law enforcement personnel in evaluating a DWI suspect's cognitive status. Because method 400 results in a quantitative analysis of the suspect's cognitive status, method 400 can provide useful evidence of a DWI infraction. Lastly, by adjusting a degree-of-difficulty associated with the physical task in accordance with the variability metric, method 400 can be used for cognitive training (e.g., in particular when a cognitive stressor is utilized as well).

One of ordinary skill will recognize that these applications, as well as the other application described through this document, are but representative samples of the possible applications of method 400.

FIGS. 5A-5C illustrate an example of a motion data acquisition scenario 500, in accordance with some embodiments. In motion data acquisition scenario 500, motion analysis is used for evaluating cognitive performance of a subject by testing movements under the subject's voluntary control, and, in particular, a subject's ability to walk regularly. In motion data acquisition scenario 500, a treadmill 502 prompts the subject to walk (e.g., perform the physical task of walking, in which each step is considered an iteration of the physical task). In doing so, the subject moves her right foot, to which an optical marker 118-8 is coupled. Cameras 112-9 and 112-10 record positional information of the optical marker 118-8 during each step, and, by extension, record positional information about the subject's right foot. The positional information is optionally processed (e.g., by computer system 110, FIG. 1) to produce (e.g., infer, or calculate) one or more positional measurements of the subject's right foot during each step. Such positional measurements optionally include position measurements (e.g., x, y, z coordinates as measured with respect to axes 508, which indicates a laboratory reference frame), acceleration measurements, velocity measurements, and/or angular measurements.

It should be understood that measurements corresponding to movement of the subject's other body parts may also be obtained; for simplicity, the present example is explained with reference to measurements corresponding to movement of the subject's right foot.

In some embodiments, subject 102 is prompted to perform the plurality of iterations of the physical task using stimuli appropriate to the task and subject. For example, the prompting can include audio stimuli (e.g., audio output of the words “Begin walking in three . . . two . . . one.”), displayed stimuli (e.g., displayed output of the words “Begin walking in three . . . two . . . one.”), and/or mechanical stimuli (e.g., treadmill 502 beginning to move).

Motion data acquisition scenario 500 is an example in which the subject is prompted to move a body part (e.g., her foot) in a periodic manner with respect to a particular reference frame of measurement (e.g., the laboratory reference frame denoted by axis 502), meaning that optimal execution of the physical task of walking on treadmill 502 would involve keeping pace with the treadmill and walking with a regular gait (e.g., a gait with a low variability). A healthy subject, one in full possession of her faculties, will be able to execute the physical task with substantially periodic movement of her right foot. In contrast, an unhealthy subject may not be able to keep pace with the treadmill, and/or may walk with an irregular gait. Such deficiencies are, in some circumstances, indicative of deficiencies in cognition and/or anticipatory timing.

FIG. 5B illustrates the path of the subject's right foot with respect to the laboratory reference frame indicated by axes 508. Each dash in the path represents a respective measurement by cameras 112 made during a subset of the steps taken by the subject. The rate at which measurements are obtained is sometimes referred to as a sampling rate. In some embodiments, the sampling rate is constant in time (e.g., sampling occurs at a predefined frequency, such as 200 Hertz (Hz), 500 Hertz (Hz), etc.). In some embodiments, the position of the foot is measured “continuously” (e.g., at a sampling rate that is at least ten times, or at least twenty times, or at least fifty times faster than the rate at which the subject completes iterations of the physical task). A constant sampling rate means that the sampling is decoupled from the physical task being performed by the subject. For contrast, consider that, in some embodiments, foot-fall times and foot-fall positions of a subject walking on a treadmill are measured and, thus, the sampling is coupled to the physical task. More generally, in some embodiments, cameras 112-9 and 112-10 (FIG. 5A) obtain a plurality of measurements for each iteration of the physical task.

FIG. 5C illustrates a nominal path of the subject's right foot determined (e.g., by a host or server system coupled to or in communication with cameras 112-9 and 112-10) with respect to the laboratory reference frame indicated by axes 508, in accordance with some embodiments. The nominal path is based at least in part on measurements obtained from a subset of the iterations of the physical task (e.g., the same subset used in FIG. 5B or, alternatively, a different subset). For example, in some embodiments, the nominal path is an average path of the first 10 iterations. Alternatively, in some embodiments, the subject is given a “training period” during which she is given time to acclimate to the task. To that end, in some implementations, a predefined number of iterations (e.g., the first 5 iterations, or the first 10 iterations, or the first 30 iterations, etc.) are ignored (e.g., measurement of which are discarded, or never obtained in the first place) in determining the nominal path. Alternatively, iterations performed during a predetermined amount of time corresponding to the training period are ignored (e.g., measurements are either discarded or not obtained for the first 5 seconds, the first 10 seconds, the first 30 seconds, etc.).

A variability metric is generated by analyzing a plurality of the measurements of the subject's right foot position shown in FIG. 5B with respect to the nominal path shown in FIG. 5C. The plurality of measurements includes at least one measurement from each iteration of a second subset of the iterations of the physical task. For example, in some embodiments, the first subset of iterations of the physical task and the second subset of iterations of the physical task both comprise all of the iterations following the training period (e.g., the first subset of iterations of the physical task is identical to the second subset of iterations of the physical task).

FIGS. 6A-6C illustrate an example of a motion data acquisition scenario 600, in accordance with some embodiments. In motion data acquisition scenario 600, motion analysis is used for evaluating cognitive performance of a subject by testing movements under the subject's voluntary control, and, in particular, a subject's ability to walk regularly. In motion data acquisition scenario 600, a portable multifunction device 604 prompts the subject to walk (e.g., perform the physical task of walking, in which each step is considered an iteration of the physical task). In doing so, the subject moves her right foot, to which a motion sensor 106-5 is coupled, and moves her left foot, to which another motion sensor 106-6 is coupled. In some embodiments, motion sensor 106-5 and motion sensor 106-6 record positional information corresponding to the subject's right foot and left foot, respectively. In some embodiments, motion sensors 106-5 and 106-6 alternatively, or in addition, transmit positional information corresponding to the subject's right foot and left foot, respectively. Portable multifunction device 604, which is in wireless communication with motion sensors 106, optionally processes the positional information to produce (e.g., infer, or calculate) one or more positional measurements of the subject's right foot during each step.

In contrast to motion data acquisition scenario 500, the positional measurements obtained in motion data acquisition scenario 600 are produced with respect to a moving reference frame co-located with the subject's left foot (e.g., the positional measurements are established in x′, y′, z′ coordinates as measured with respect to axes 608, which indicates a reference frame co-located with the subject's left foot). The positional measurements optionally include position measurements, acceleration measurements, velocity measurements, and/or angular measurements.

In some embodiments, portable multifunction device 604 prompts the subject to perform the physical task (i.e., start walking) at the beginning of a motion data acquisition scenario. For example, the subject will execute a mobile application (“app”) on portable multifunction device 604 that prompts the user with the message (e.g., audibly and/or visually), “Whenever you're ready, begin walking in a straight line.” Alternatively, in some embodiments, portable multifunction device 604 detects (e.g., automatically, without the subject's intervention) when sensor 106-5 is undergoing substantially periodic motion with respect to a reference frame co-located with motion sensor 106-6. To this end, in some embodiments, portable multifunction device 604 is configured to execute an algorithm designed to infer whether a first body part is undergoing substantially periodic motion with respect to a second body part (e.g., walking in a straight line, in which case the first body part is one foot and the second body part is the other foot). Such an algorithm can be realized, for example, by determining a temporal period of movement using a fast Fourier transform (FFT) and analyzing movement consistency from temporal period to temporal period. Thus, in some implementations, portable multifunction device 604 ignores random foot movements, such as those made while the subject is sitting at a desk, either by determining that a periodicity metric is not strong enough (in accordance with an FFT intensity) or by determining that the foot movements are not consistent enough from temporal period to temporal period.

In such circumstances, the prompt may occur well before the motion data acquisition scenario. For example, the subject may begin executing the mobile app at the beginning of the day, at which point the subject will receive instructions from portable multifunction device 604 indicating that the mobile app will analyze her walking patterns whenever possible (e.g., based upon predefined criteria through which a determination is made that the subject is walking in a straight line). In this manner, portable multifunction device 604 samples the subject's cognitive state throughout the day without the subject intervening or intentionally performing the physical task. Portable multifunction device 604 can then alert the subject—who may be, for example, a diabetic—of subtle changes in subject's cognitive state before she would otherwise be aware of the problem.

FIG. 6B illustrates the path of the subject's right foot with respect to the moving reference frame indicated by axes 608. Each dash in the path represents a respective relative measurement by motion sensors 106-5 and 106-6 made during a subset of the steps taken by the subject (e.g., a measurement of the location of motion sensor 106-5 relative to the moving reference frame co-located with motion sensor 106-6). FIG. 6B is otherwise analogous to FIG. 5B.

FIG. 6C illustrates a nominal path of the subject's right foot determined (e.g., by portable multifunction device 604, or by a host system remotely located with respect to portable multifunction device 604) with respect to the moving reference frame indicated by axes 608, in accordance with some embodiments. FIG. 6C is otherwise analogous to FIG. 5C.

FIGS. 7A-7C illustrate an example of a motion data acquisition scenario 700, in accordance with some embodiments. In motion data acquisition scenario 700, motion analysis is used for evaluating cognitive performance of a subject by testing movements under the subject's voluntary control, in particular, the subject's ability to visually track a smoothly moving object. In motion data acquisition scenario 700, a subject is prompted to follow, with her eyes, a smoothly moving image 703 (e.g., a dot or ball moving at a constant speed) that follows a predefined path (e.g., a circular or oval path) on a display 120. Each repetition of the user visually tracking the smoothly moving object around the predictable path is considered an iteration of the physical task. One or more cameras 112 are focused on the subject's eyes so that eye positions (and, in some embodiments, eye movements) of the subject are measured with respect to a laboratory reference frame indicated by axes 708. In some implementations, the one or more cameras 112 are mounted on the subject's head by head equipment 722 (e.g., a headband, helmet, or pair of glasses). Various mechanisms, optionally, stabilize the subject's head (for example, to keep the distance between the subject and display 120 fixed), and maintain the orientation of subject's head. In some embodiments, the distance between the subject and display 120 is kept fixed at approximately 40 cm. In some implementations, head equipment 722 includes the head equipment and apparatuses described in U.S. Patent Publication 2010/0204628 A1, which is incorporated by reference in its entirety.

FIG. 7B illustrates the path of the subject's left eye and the path of the subject's right eye with respect to the laboratory reference frame indicated by axes 708. Each dash in the path represents a respective measurement by a camera 112 made during a subset of the iterations of the image 703 traversing the predefined path. FIG. 7B is otherwise analogous to FIG. 5B.

FIG. 7C illustrates nominal paths of the subject's left eye and the subject's right eye, respectively, with respect to the laboratory reference frame indicated by axes 708, in accordance with some embodiments. FIG. 7C is otherwise analogous to FIG. 5C.

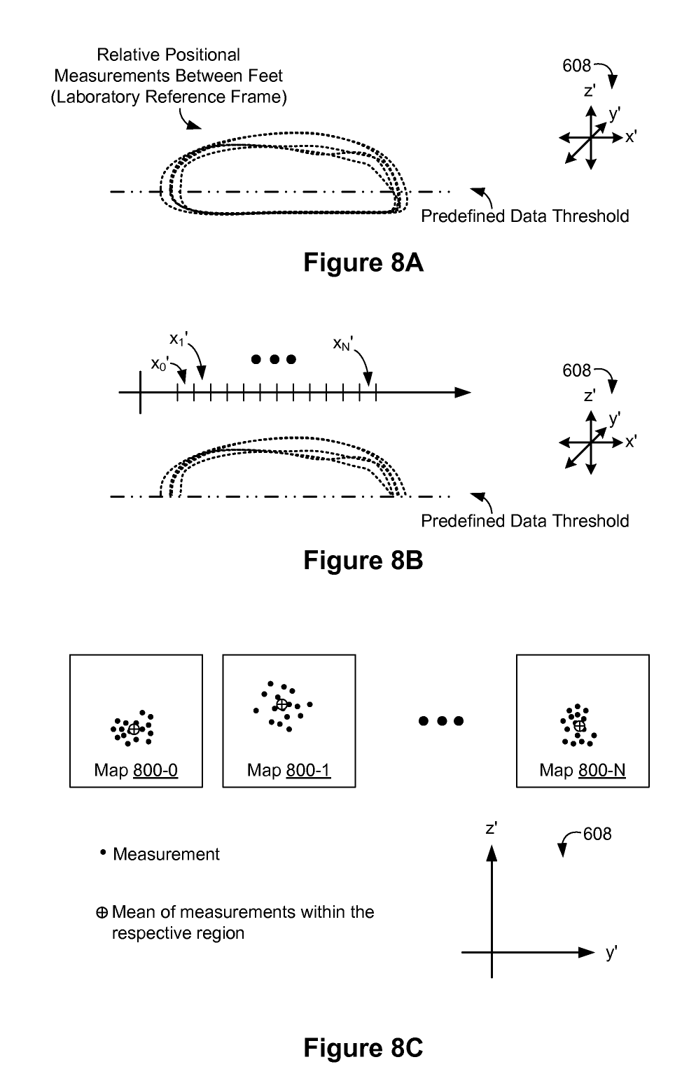

FIGS. 8A-8C illustrate motion analysis used to generate a variability metric, in accordance with some embodiments. The motion analysis is used to generate a variability metric for evaluating cognitive performance of a subject by testing movements under the subject's voluntary control. In particular, as an example, FIG. 8A illustrates an example of motion analysis performed using the measurements obtained during motion data acquisition scenario 600 in FIGS. 6A-6C.

In FIG. 8A, measurements obtained during motion data acquisition scenario 600 in FIGS. 6A-6C are shown, along with a predefined data threshold. In this example, data with a z′ value (as indicated by axes 608) less than the predefined data threshold are discarded, as shown in FIG. 8B, resulting in a subset of the measurements to be used in the motion analysis. Furthermore, as shown in FIG. 8B, the x′ axis is partitioned into a plurality of regions, such as x₀′, x₁′ . . . x_{N}′. Each measurement in the subset of measurements is assigned to a corresponding partition to form a plurality of maps 800 (e.g., map 800-0 corresponding to region x₀′, map 800-1 corresponding to region x₁′, through map 800-N corresponding to region x_{N}′). A variability value is generated by calculating an average distance from each respective measurement in the subset of measurements to a mean location of measurements in a respective region that corresponds to the respective measurement. For example, in some embodiments, a variability metric G generating using the equation:

$$G = \frac{1}{M} {\sum }_{i = 0}^{N} {\sum }_{j = 1}^{{K}_{i}} {( \vert {\overline{r}}_{j} - {\overline{μ}}_{i} \vert )}^{2}$$

In this equation, N is a count of regions, r̅ⱼ is a respective measurement in an i^{th} region, Kᵢ is a count of measurements in the i^{th} region, μ̅ᵢ is a mean location of measurements in the i^{th} region, and M is a count of measurements in the subset of measurements (e.g., M=Σᵢ₌₀^{N}Kᵢ). In this example, the nominal path is determined implicitly as the set of mean locations of measurements μ̅ᵢ.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

It will be understood that, although the terms “first,” “second,” etc. may be used herein to describe various elements, these elements should not be limited by these terms. These terms are only used to distinguish one element from another. For example, a first sound detector could be termed a second sound detector, and, similarly, a second sound detector could be termed a first sound detector, without changing the meaning of the description, so long as all occurrences of the “first sound detector” are renamed consistently and all occurrences of the “second sound detector” are renamed consistently. The first sound detector and the second sound detector are both sound detectors, but they are not the same sound detector.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “upon a determination that” or “in response to determining” or “in accordance with a determination” or “upon detecting” or “in response to detecting” that the stated condition precedent is true, depending on the context.

What is claimed is:

1. A method of evaluating cognitive performance of a subject by testing movements under the subject's voluntary control, comprising:
prompting the subject to perform a plurality of iterations of a physical task, wherein the physical task includes a movement of a first body part of the subject's body;
for each iteration of the plurality of iterations of the physical task, obtaining one or more measurements corresponding to the movement of the first body part;
determining a nominal path of the first body part based on the measurements obtained from a first subset of the plurality of iterations of the physical task, the first subset of the plurality of iterations of the physical task comprising some or all of said plurality of iterations of the physical task;
generating a variability metric by analyzing a plurality of measurements with respect to the nominal path, wherein the plurality of measurements includes at least one measurement from each iteration of a second subset of the plurality of iterations of the physical task; and
comparing the variability metric with a predetermined baseline to categorize the cognitive performance of the subject.

2. The method of claim 1, wherein, for each iteration of the plurality of iterations of the physical task, the one or more measurements corresponding to the movement of the first body part include two or more measurements corresponding to the movement of the first body part.

3. The method of claim 1, wherein the variability metric corresponds to variability of error with respect to the nominal path.

4. The method of claim 1, wherein the plurality of measurements includes a plurality of positional measurements, each positional measurement of the plurality of positional measurements being one of a position measurement, a velocity measurement, or an acceleration measurement, and each positional measurement including one or more positional values corresponding to the first body part.

5. The method of claim 4, wherein each positional value corresponds to a direction in accordance with a reference frame of measurement, the reference frame of measurement being one of a laboratory reference frame, a moving reference frame co-located with a target, or a moving reference frame co-located with a second body part.

6. The method of claim 5, wherein prompting the subject to perform the plurality of iterations of the physical task includes prompting the subject to move the first body part in a periodic manner with respect to the reference frame of measurement.

7. The method of claim 1, wherein prompting the subject to perform the plurality of iterations of the physical task includes one of:
prompting the subject to track a periodically moving target on a display, wherein said tracking is performed by the subject with a respective part of the subject's body and each iteration corresponds to a respective period of the periodic movement of the target;
prompting the subject to walk a plurality of steps, wherein each step corresponds to a respective iteration of the plurality of iterations; and
prompting the subject to repeatedly reach with a respective hand toward an object, wherein each iteration corresponds to an instance of the subject reaching toward the object with the respective hand.

8. The method of claim 1, wherein:
the predetermined baseline corresponds to a predefined degree of variability in performing the physical task; and
comparing the variability metric with the predetermined baseline to categorize the cognitive performance of the subject includes:
categorizing the variability metric as indicative of a higher cognitive performance when the variability metric corresponds to a lower degree of variability in performing the physical task than the predefined degree of variability; and
categorizing the variability metric as indicative of a lower cognitive performance when the variability metric corresponds to a higher degree of variability in performing the physical task than the predefined degree of variability.

9. The method of claim 1, further including prompting the subject to concurrently perform a second task while performing the physical task, wherein the second task is a cognitive stressor.

10. The method of claim 1, wherein:
determining the nominal path of the first body part based on the measurements obtained from the first subset of the plurality of iterations of the physical task includes calculating a dynamical periodic orbit of motion, wherein the dynamical periodic orbit of motion represents an average movement of the first body part over the first subset of the plurality of iterations of the physical task; and
generating the variability metric includes calculating a value corresponding to an average distance between the measurements obtained from the second subset of the plurality of iterations of the physical task and the dynamical periodic orbit of motion.

11. The method of claim 1, wherein generating the variability metric by analyzing the plurality of measurements further includes:
producing a plurality of comparison values by, for each iteration of the second subset of the plurality of iterations of the physical task, comparing a respective measurement to a corresponding previous measurement from a previous iteration of the plurality of iterations to produce a respective comparison value; and
generating the variability metric by aggregating the respective comparison values from each of the second subset of the plurality of iterations of the physical task.

12. The method of claim 1, wherein generating the variability metric by analyzing the plurality of measurements includes performing one or more of: a return map analysis, a variance analysis, a covariance analysis, a principal component analysis, an independent component analysis, a K-means analysis, a medoid-based analysis, a dependency analysis, an entropy analysis, and a multi-resolution analysis.

13. The method of claim 1, wherein the predetermined baseline is based on at least one of:
a variability range associated with a preselected group of control subjects;
a demographic of the subject; and
a variability metric for the subject generated from a previous test.

14. A system for evaluating cognitive performance of a subject by testing movements under the subject's voluntary control, comprising:
one or more processors;
memory; and
one or more programs stored in the memory, the one or more programs comprising instructions to:
prompt the subject to perform a plurality of iterations of a physical task, wherein the physical task includes a movement of a first body part of the subject's body;
for each iteration of the plurality of iterations of the physical task, obtain one or more measurements corresponding to the movement of the first body part;
determine a nominal path of the first body part based on the measurements obtained from a first subset of the plurality of iterations of the physical task, the first subset of the plurality of iterations of the physical task comprising some or all of said plurality of iterations of the physical task;
generate a variability metric by analyzing a plurality of measurements with respect to the nominal path, wherein the plurality of measurements includes at least one measurement from each iteration of a second subset of the plurality of iterations of the physical task; and
compare the variability metric with a predetermined baseline to categorize the cognitive performance of the subject.

15. The system of claim 14, wherein, for each iteration of the plurality of iterations of the physical task, the one or more measurements corresponding to the movement of the first body part include two or more measurements corresponding to the movement of the first body part.

16. The system of claim 14, wherein the variability metric corresponds to variability of error with respect to the nominal path.

17. The system of claim 14, wherein the plurality of measurements includes a plurality of positional measurements, each positional measurement of the plurality of positional measurements being one of a position measurement, a velocity measurement, or an acceleration measurement, and each positional measurement including one or more positional values corresponding to the first body part.

18. The system of claim 17, wherein each positional value corresponds to a direction in accordance with a reference frame of measurement, the reference frame of measurement being one of a laboratory reference frame, a moving reference frame co-located with a target, or a moving reference frame co-located with a second body part.

19. The system of claim 18, wherein the instructions to prompt the subject to perform the plurality of iterations of the physical task include instructions to prompt the subject to move the first body part in a periodic manner with respect to the reference frame of measurement.

20. The system of claim 14, wherein the instructions to prompt the subject to perform the plurality of iterations of the physical task include instructions to prompt the subject in at least one of the following ways:
prompt the subject to track a periodically moving target on a display, wherein said tracking is performed by the subject with a respective part of the subject's body and each iteration corresponds to a respective period of the periodic movement of the target;
prompt the subject to walk a plurality of steps, wherein each step corresponds to a respective iteration of the plurality of iterations; and
prompt the subject to repeatedly reach with a respective hand toward an object, wherein each iteration corresponds to an instance of the subject reaching toward the object with the respective hand.

21. The system of claim 14, wherein:
the predetermined baseline corresponds to a predefined degree of variability in performing the physical task; and
the instructions to compare the variability metric with the predetermined baseline to categorize the cognitive performance of the subject include instructions to:
categorize the variability metric as indicative of a higher cognitive performance when the variability metric corresponds to a lower degree of variability in performing the physical task than the predefined degree of variability; and
categorize the variability metric as indicative of a lower cognitive performance when the variability metric corresponds to a higher degree of variability in performing the physical task than the predefined degree of variability.

22. The system of claim 14, further including instructions to prompt the subject to concurrently perform a second task while performing the physical task, wherein the second task is a cognitive stressor.

23. The system of claim 14, wherein:
the instructions to determine the nominal path of the first body part based on the measurements obtained from the first subset of the plurality of iterations of the physical task include instructions to calculate a dynamical periodic orbit of motion, wherein the dynamical periodic orbit of motion represents an average movement of the first body part over the first subset of the plurality of iterations of the physical task; and
the instructions to generate the variability metric include instructions to calculate a value corresponding to an average distance between the measurements obtained from the second subset of the plurality of iterations of the physical task and the dynamical periodic orbit of motion.

24. The system of claim 14, wherein the instructions to generate the variability metric by analyzing the plurality of measurements further include instructions to:
produce a plurality of comparison values by, for each iteration of the second subset of the plurality of iterations of the physical task, comparing a respective measurement to a corresponding previous measurement from a previous iteration of the plurality of iterations to produce a respective comparison value;
generate the variability metric by aggregating the respective comparison values from each of the second subset of the plurality of iterations of the physical task.

25. The system of claim 14, wherein the instructions to generate the variability metric by analyzing the plurality of measurements include instructions to perform one or more of: a return map analysis, a variance analysis, a covariance analysis, a principal component analysis, an independent component analysis, a K-means analysis, a medoid-based analysis, a dependency analysis, an entropy analysis, and a multi-resolution analysis.

26. The system of claim 14, wherein the predetermined baseline is based on at least one of:
a variability range associated with a preselected group of control subjects;
a demographic of the subject; and
a variability metric for the subject generated from a previous test.

27. A non-transitory computer readable storage medium storing one or more programs, the one or more programs comprising instructions to:
prompt the subject to perform a plurality of iterations of a physical task, wherein the physical task includes a movement of a first body part of the subject's body;
for each iteration of the plurality of iterations of the physical task, obtain one or more measurements corresponding to the movement of the first body part;
determine a nominal path of the first body part based on the measurements obtained from a first subset of the plurality of iterations of the physical task, the first subset of the plurality of iterations of the physical task comprising some or all of said plurality of iterations of the physical task;
generate a variability metric by analyzing a plurality of measurements with respect to the nominal path, wherein the plurality of measurements includes at least one measurement from each iteration of a second subset of the plurality of iterations of the physical task; and
compare the variability metric with a predetermined baseline to categorize the cognitive performance of the subject.
